(19) United States
(12) Patent Application Publication
(10) Pub. No.: US 2024/0216093 A1
(43) Pub. Date: Jul. 4, 2024

(54) INTEGRATION OF FORCE ISOLATION ELEMENTS INTO STEERABLE ELONGATE MEMBER

(71) Applicant: Auris Health, Inc., Redwood City, CA (US)

(72) Inventors: Taylor E. Furtado, Campbell, CA (US); James Hayes, La Honda, CA (US); Justin M. Gilbert, San Francisco, CA (US); Sean N. Finson, Santa Clara, CA (US)

(21) Appl. No.: 18/536,621

(22) Filed: Dec. 12, 2023

Related U.S. Application Data
(60) Provisional application No. 63/436,194, filed on Dec. 30, 2022.

(51) Int. Cl. A61B 34/00; A61B 1/005; A61B 1/307; A61B 17/221; A61B 34/37

(52) U.S. Cl. CPC A61B 34/71; A61B 1/0057; A61B 1/307; A61B 17/221; A61B 34/37; A61B 2017/2215

(57) ABSTRACT

An apparatus includes an elongate body and a tendon assembly. The elongate body includes a sidewall comprising a flexible material, a proximal portion, and a distal portion. The tendon assembly is operable to drive deflection of a portion of the elongate body away from the central longitudinal axis. The tendon assembly includes a tendon housing and a tendon. The tendon extends through the sidewall. The tendon housing has a distal end secured at a longitudinal position along the elongate body. The tendon is slidably disposed in the tendon housing. The tendon has a distal portion extending distally from the distal end of the tendon housing. The distal portion of the tendon is fixedly secured relative to the elongate body.

### PRIORITY

This application claims the benefit of U.S. Pat. App. No. 63/436,194, entitled “Integration of Force Isolation Elements into Steerable Elongate Member,” filed Dec. 30, 2022, the disclosure of which is incorporated by reference herein.

### BACKGROUND

A variety of surgical instruments include an end effector for use in medical treatments and procedures conducted by a medical professional operator, including applications in robotically assisted surgeries. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient); or quite near the patient in the operating room. The controller may include one or more hand input devices (e.g., joysticks, exoskeletal gloves, master manipulators, etc.), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probes, etc. Each of these structures performs functions for the surgeon, for example, cutting tissue, coagulating tissue, manipulating a needle, grasping a blood vessel, dissecting tissue, or cauterizing tissue. A robotically-controlled instrument may be introduced into the patient via an incision, via a naturally occurring orifice, or otherwise.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a top plan view of an example of a robotic surgical system being used in a urological procedure.

FIG. 2 depicts a schematic view of different components of the robotic surgical system of FIG. 1.

FIG. 3 depicts enlarged views of other components of the robotic surgical system of FIG. 1, including a distal portion of a ureteroscope.

FIG. 4 depicts a schematic view of an example of an articulating elongate member that may be used with the robotic surgical system of FIG. 1.

FIG. 5 depicts a cross-sectional side view of the elongate member of FIG. 4, taken along line 5-5 of FIG. 4.

FIG. 6 depicts a cross-sectional end view of the elongate member of FIG. 5, taken along line 6-6 of FIG. 5.

FIG. 7 depicts a cross-sectional end view of the elongate member of FIG. 5, taken along line 7-7 of FIG. 5.

FIG. 8 depicts a cross-sectional end view of the elongate member of FIG. 5, taken along line 8-8 of FIG. 5.

FIG. 9 depicts a cross-sectional end view of the elongate member of FIG. 5, taken along line 9-9 of FIG. 5.

FIG. 10 depicts a perspective view of components of an apparatus that may be used to manufacture an elongate member having a braided structure.

FIG. 11 depicts an example of a mandrel set that may be used in a variation of the apparatus of FIG. 10 to manufacture the elongate member of FIG. 5.

FIG. 12 depicts a cross-sectional end view of the mandrel set of FIG. 11, taken along line 12-12 of FIG. 11.

FIG. 13A depicts a cross-sectional end view of the mandrel set of FIG. 11, taken along line 12-12 of FIG. 11, as the mandrel set is being used in a first portion of a process of manufacturing the elongate member of FIG. 5.

FIG. 13B depicts a cross-sectional end view of the mandrel set of FIG. 11, taken along line 12-12 of FIG. 11, as the mandrel set is being used in a second portion of a process of manufacturing the elongate member of FIG. 5.

FIG. 13C depicts a cross-sectional end view of the mandrel set of FIG. 11, taken along line 12-12 of FIG. 11, as the mandrel set is being used in a third portion of a process of manufacturing the elongate member of FIG. 5.

FIG. 13D depicts a cross-sectional end view of a portion of the elongate member of FIG. 5 after the remaining mandrels of the mandrel set of FIG. 11 have been removed during a process of manufacturing the elongate member of FIG. 5.

FIG. 14 depicts a schematic view of examples of components and steps that may be used to perform a process of manufacturing the elongate member of FIG. 5.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

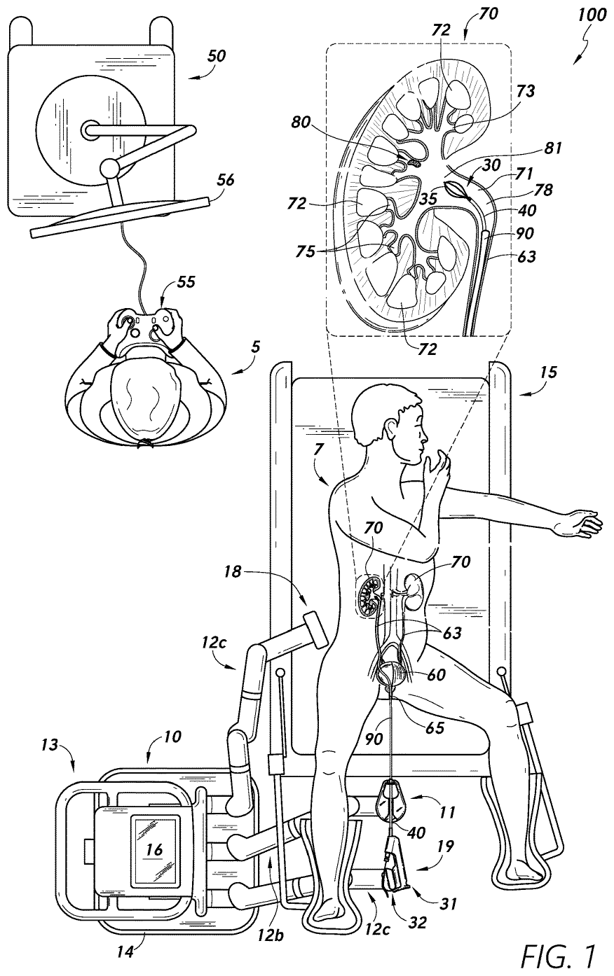

FIG. 1 shows an example medical system (100) for performing various medical procedures in accordance with aspects of the present disclosure. The medical system (100) may be used for, for example, endoscopic (e.g., ureteroscopic) procedures. Certain ureteroscopic procedures involve the treatment/removal of kidney stones. Although the system (100) of FIG. 1 is presented in the context of a ureteroscopic procedure, it should be understood that the principles disclosed herein may be implemented in any type of endoscopic (e.g., bronchial, gastrointestinal, etc.) and/or percutaneous procedure.

The medical system (100) of the present includes a robotic system (10) (e.g., mobile robotic cart) that is configured to engage with and/or control one or more medical instruments (e.g., ureteroscope (40), basketing system (30), etc.) via one or more robotic arms (12) to perform a direct-entry procedure on a patient (7). In some versions, the robotic system (10) and/or control system (50) is/are configured to receive images and/or image data from the scope (40) representing internal anatomy of the patient (7), namely the urinary system with respect to the particular depiction of FIG. 1, and/or display images based thereon.

It should be understood that the direct-entry instrument(s) operated through systems (10, 50) may include any type of medical instrument or combination of instruments, including an endoscope (such as a ureteroscope (40)), catheter (such as a steerable or non-steerable catheter), nephroscopes, laparoscope, basketing systems (30), and/or other type of medical instrument(s). The various scope-type instruments disclosed herein, such as the scope (40) of the system (100), may be configured to navigate within the human anatomy, such as within a natural orifice or lumen of the human anatomy. The terms “scope” and “endoscope” are used herein according to their broad and ordinary meanings; and may refer to any type of elongate medical instrument having image generating, viewing, and/or capturing functionality and configured to be introduced into any type of organ, cavity, lumen, chamber, or space of a body. A scope may include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise a rigid or flexible tube, and may be dimensioned to be passed within an outer sheath, catheter, introducer, or other lumen-type device, or may be used without such devices.

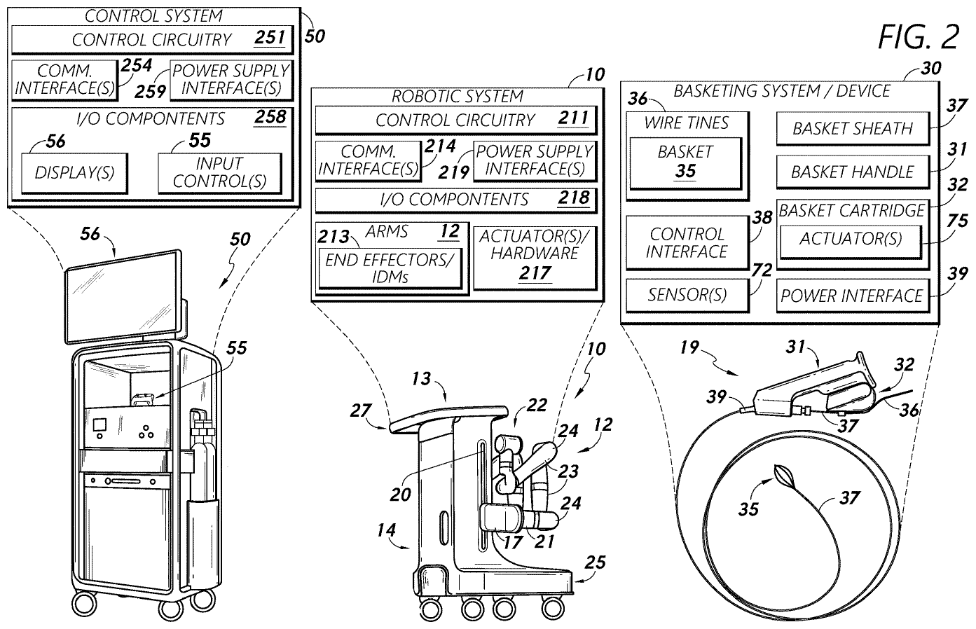

The medical system (100) of the present example further includes a control system (50), a table (15), and an electromagnetic (EM) field generator (18). Table (15) is configured to hold the patient (7). EM field generator (18) may be held by one or more of the robotic arms (12) of the robotic system (10) or may be a stand-alone device. As shown in FIGS. 1-2, control system (50) of the present example includes various input/output (I/O) components (258) configured to assist the physician (5) or others in performing a medical procedure. For example, the I/O components (258) may be configured to allow for user input to control/navigate the scope (40) and/or basketing system (30) within the patient (7). I/O components (258) of the present example include a controller (55) that is configured to receive user input from the operator; and a display (56) that is configured to present certain information to assist the operator. Controller (55) may take any suitable form, including but not limited to one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, etc.

As also shown in FIG. 2, control system (50) of the present example includes a communication interface (254) that is operable to provide a communicative interface between control system (50) and robotic system (10), basketing system (30), scope (40), and/or other components. Communications via communication interface (254) may include data, commands, electrical power, and/or other forms of communication. Communication interface (254) may also be configured to provide communication via wire, wirelessly, and/or other modalities. Control system (50) also includes a power supply interface (259), which may receive power to drive control system (50) via wire, battery, and/or any other suitable kind of power source. A control circuitry (251) of control system (50) may provide signal processing and execute control algorithms to achieve the functionality of medical system (100) as described herein.

The control system (50) may also communicate with the robotic system (10) to receive position data therefrom relating to the position of the distal end of the scope (40), access sheath (90), or basketing device (30). Such positional data relating to the position of the scope (40), access sheath (90), or basketing device (30) may be derived using one or more electromagnetic sensors associated with the respective components. Moreover, in some versions, the control system (50) may communicate with the table (15) to position the table (15) in a particular orientation or otherwise control the table (15). The control system (50) may also communicate with the EM field generator (18) to control generation of an EM field in an area around the patient (7).

As noted above and as shown in FIGS. 1-2, robotic system (10) includes robotic arms (12) that are configured to engage with and/or control scope (40) and/or the basketing system (30) to perform one or more aspects of a procedure. It should be understood that robotic arms (12) may be coupled to different instruments than what is shown in FIG. 1; and in some scenarios, one or more of the robotic arms (12) may not be utilized or coupled to a medical instrument. Each robotic arm (12) includes multiple arm segments (23) coupled to joints (24), which may provide multiple degrees of movement/freedom. In the example of FIG. 1, the robotic system (10) is positioned proximate to the patient's legs and the robotic arms (12) are actuated to engage with and position the scope (40) for access into an access opening, such as the urethra (65) of the patient (7). When the robotic system (10) is properly positioned, the scope (40) may be inserted into the patient (7) robotically using the robotic arms (12), manually by the physician (5), or a combination thereof. A scope-driver instrument coupling (11) (e.g., instrument device manipulator (IDM)) may be attached to the distal portion of one of the arms (12b) to facilitate robotic control/advancement of the scope (40). Another (12c) of the arms may include an instrument coupling/manipulator (19) that is configured to facilitate advancement and operation of the basketing device (30). The scope (40) may include one or more working channels through which additional tools, such as lithotripters, basketing devices, forceps, etc., may be introduced into the treatment site.

The robotic system (10) may be coupled to any component of the medical system (100), such as the control system (50), the table (15), the EM field generator (18), the scope (40), the basketing system (30), and/or any type of percutaneous-access instrument (e.g., needle, catheter, nephroscope, etc.). As noted above, robotic system (10) may be communicatively coupled with control system (50) via communication interfaces (214, 254). Robotic system (10) also includes a power supply interface (219), which may receive power to drive robotic system (10) via wire, battery, and/or any other suitable kind of power source. In addition, robotic system (10) of the present example includes various input/output (I/O) components (218) configured to assist the physician (5) or others in performing a medical procedure. Such I/O components (218) may include any of the various kinds of I/O components (258) described herein in the context of control system (50). In addition, or in the alternative, I/O components (218) of robotic system (10) may take any suitable form (or may be omitted altogether).

Robotic system (10) of the present example generally includes a column (14), a base (25), and a console (13) at the top of the column (14). The column (14) may include one or more arm supports (17) (also referred to as a “carriage”) for supporting the deployment of the one or more robotic arms (12) (three shown in FIG. 2). The arm support (17) may include individually-configurable arm mounts that rotate along a perpendicular axis to adjust the base of the robotic arms (12) for desired positioning relative to the patient. In some versions, the arm support (17) may be connected to the column (14) through slots (20) that are positioned on opposite sides of the column (14) to guide vertical translation of the arm support (17) along column (14). The robotic arms (12) of the present example generally comprise robotic arm bases (21) and end effectors (22), separated by a series of linking arm segments (23) that are connected by a series of joints (24), each joint comprising one or more independent actuators (217). Each actuator (217) may comprise an independently-controllable motor. I/O components (218) may be positioned at the upper end of column (14). Console (13) also includes a handle (27) to assist with maneuvering and stabilizing robotic system (10).

The end effector (213) of each of the robotic arms (12) may include an instrument device manipulator (IDM), which may be attached using a mechanism changer interface (MCI). In some versions, the IDM (213) may be removed and replaced with a different type of IDM (213), for example, a first type (11) of IDM (213) may manipulate a scope (40), while a second type (19) of IDM (213) may manipulate a basketing system (30). Another type of IDM (213) may be configured to hold an electromagnetic field generator (18). An MCI may provide power and control interfaces (e.g., connectors to transfer pneumatic pressure, electrical power, electrical signals, and/or optical signals from the robotic arm (12) to the IDM (213). The IDMs (213) may be configured to manipulate medical instruments (e.g., surgical tools/instruments), such as the scope (40), using techniques including, for example, direct drives, harmonic drives, geared drives, belts and pulleys, magnetic drives, and the like.

The system (100) may include certain control circuitry configured to perform certain of the functionality described herein, including the control circuitry (211) of the robotic system (10) and the control circuitry (251) of the control system (50). That is, the control circuitry of the system (100) may be part of the robotic system (10), the control system (50), or some combination thereof. The term “control circuitry” is used herein according to its broad and ordinary meaning, and may refer to any collection of processors, processing circuitry, processing modules/units, chips, dies (e.g., semiconductor dies including come or more active and/or passive devices and/or connectivity circuitry), microprocessors, micro-controllers, digital signal processors, microcomputers, central processing units, field programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in versions in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry (211, 251) may comprise computer-readable media storing, and/or configured to store, hard-coded and/or operational instructions corresponding to at least some of the steps and/or functions illustrated in one or more of the present figures and/or described herein. Such computer-readable media may be included in an article of manufacture in some instances. The control circuitry (211, 251) may be entirely locally maintained/disposed or may be remotely located at least in part (e.g., communicatively coupled indirectly via a local area network and/or a wide area network).

In some versions, for example, the physician (5) may provide input to the control system (50) and/or robotic system (10); and in response to such input, control signals may be sent to the robotic system (10) to manipulate the scope (40) and/or catheter basketing system (30). The control system (50) may include one or more display devices (56) to provide various information regarding a procedure. For example, the display(s) (56) may provide information regarding the scope (40) and/or basketing system (30). The control system (50) may receive real-time images that are captured by the scope (40) and display the real-time images via the display(s) (56).

As shown in FIG. 2, the basketing device (30) of the present example includes a basket (35) formed of one or more wire tines (36) disposed within a basketing sheath (37) over a length thereof, where the tines project from a distal end of the sheath (37) to form the basket (35). The tines (36) further extend from the proximal end of the sheath (37) and are slidable within the basketing sheath (37). The tines (36) and the sheath (37) may be coupled to respective actuators (75) of a basket cartridge component (32). The basket cartridge (32) may be physically and/or communicatively coupled to a handle portion/component (31) of the basketing system (30). The handle component (31) may be configured to be used to assist in basketing control either manually or through robotic control. The basketing system (30) may be powered through a power interface (39) and/or controlled through a control interface (38), each or both of which may interface with a robotic arm/component of the robotic system (10). The basketing system (30) may further comprise one or more sensors (72), such as pressure and/or other force-reading sensors, which may be configured to generate signals indicating forces experienced at/by one or more of the actuators (75) and/or other couplings of the basketing system (30).

In an example of a use case, if the patient (7) has a kidney stone (80) located in the kidney (70), the physician may execute a procedure to remove the stone (80) through the urinary tract (65, 60, 63). In particular, and as shown in FIG. 1, the physician may operate medical system (100) to achieve direct entry of the scope (40) into the urinary tract (65, 60, 63) of the patient (7) via the urethra (65). The physician (5) may interact with the control system (50) and/or the robotic system (10) to cause/control the robotic system (10) to advance and navigate the scope (40) from the urethra (65), through the bladder (60), up the ureter (63), and into the renal pelvis (71) and/or calyx network of the kidney (70) where the stone (80) is located. The physician (5) may further interact with the control system (50) and/or the robotic system (10) to cause/control the advancement of a basketing device (30) through a working channel of the scope (40), where the basketing device (30) is configured to facilitate capture and removal of a kidney stone. The control system (50) may provide information via the display(s) (56) that is associated with the medical instrument (40), such as real-time endoscopic images captured therewith, and/or other instruments of the system (100), to assist the physician (5) in navigating/controlling such instrumentation.

In the present example, a ureteral access sheath (90) is disposed within the urinary tract (65, 60, 63) to an area near the kidney (70). The scope (40) may be passed through the ureteral access sheath (90) to gain access to the internal anatomy of the kidney (70), as shown. Once at the site of the kidney stone (80) (e.g., within a target calyx (73) of the kidney (70) through which the stone (80) is accessible), the scope (40) may be used to channel/direct the basketing device (30) to the target location. Once the stone (80) has been captured in the distal basket portion (35) of the basketing device (30), the utilized ureteral access path may be used to extract the kidney stone (80) from the patient (7).

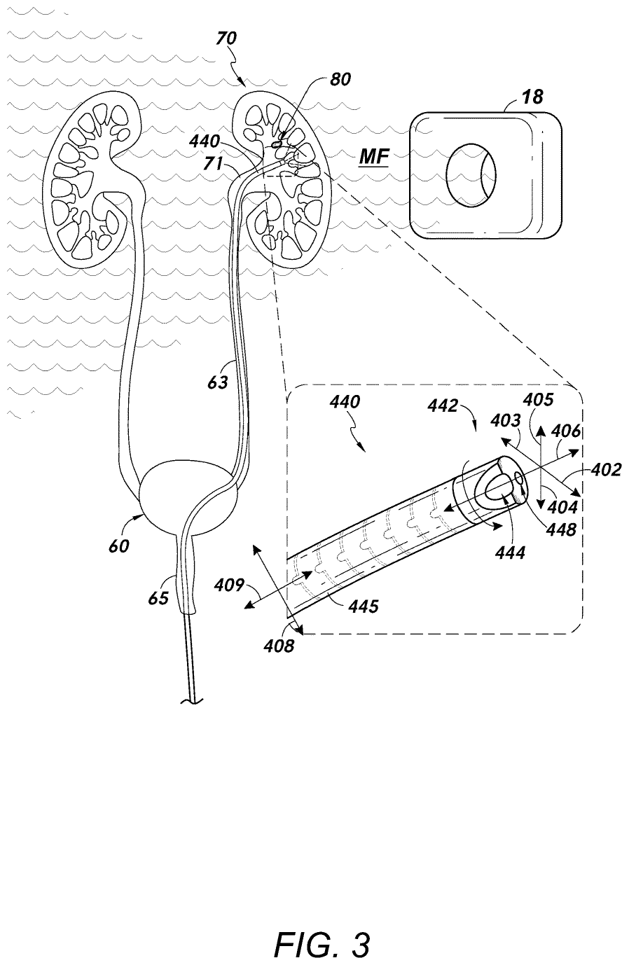

FIG. 3 shows an example of a scope (440) that may be used as scope (40) described above. Scope (440) of this example includes a working channel (444) for deploying medical instruments (e.g., lithotripters, basketing system (30), forceps, etc.), irrigation, and/or aspiration to an operative region at a distal end of the scope (440). The scope (440) may be articulated, such as with respect to at least a distal portion of the scope (440), so that the scope (440) can be steered within the human anatomy. In some versions, the scope (440) is configured to be articulated with, for example, five degrees of freedom, including XYZ coordinate movement, as well as pitch and yaw. In some versions, the scope (440) provides six degrees of freedom, including X, Y, and Z ordinate positions, as well as pitch, roll, and yaw. Position sensor(s) of the scope (440) may likewise have similar degrees of freedom with respect to the position information they produce/provide. As shown in FIG. 3, the tip (442) of the scope (440) may be oriented with zero deflection relative to a longitudinal axis (406) thereof (also referred to as a “roll axis”).

In the present example, the scope (440) can accommodate wires and/or optical fibers to transfer signals to/from an optical assembly and a distal end (442) of the scope (440), which can include an imaging device (448), such as an optical camera. The imaging device (448) may be used to capture images of an internal anatomical space, such as a target calyx/papilla of the kidney (70). The scope (440) may further be configured to accommodate optical fibers to carry light from proximately-located light sources, such as light-emitting diodes, to the distal end (442) of the scope (440). The distal end (442) of the scope (440) may include ports for light sources to illuminate an anatomical space when using the imaging device (448). The imaging device (448) may comprise an optical fiber, fiber array, and/or lens; or a light-emitting diode at distal end (442). The optical components of imaging device (448) move along with the distal end (442) of the scope (440), such that movement of the distal end (442) of the scope (440) results in changes to the images captured by the imaging device(s) 448.

To capture images at different orientations of the tip (442), robotic system (10) may be configured to deflect the tip (442) on a positive yaw axis (402), negative yaw axis (403), positive pitch axis (404), negative pitch axis (405), or roll axis (406). The tip (442) or body (445) of the scope (442) may be elongated or translated in the longitudinal axis (406), x-axis (408), or y-axis (409). The scope (440) may include a reference structure (not shown) to calibrate the position of the scope (440). For example, robotic system (10) and/or control system (50) may measure deflection of the scope (440) relative to the reference structure. The reference structure may be located, for example, on a proximal end of the endoscope (440) and may include a key, slot, or flange.

A robotic arm (12) of robotic system (10) may be configured/configurable to manipulate the scope (440) as described above. Such manipulation may be performed by actuating one or more elongate members such as one or more pull wires (e.g., pull or push wires), cables, fibers, and/or flexible shafts. For example, robotic arms (12) may be configured to actuate multiple pull wires (not shown) coupled to the scope (440) to deflect the tip (442) of the scope (440). Pull wires may include any suitable or desirable materials, such as metallic and non-metallic materials such as stainless steel, aramid fiber, tungsten, carbon fiber, and the like. In some versions, the scope (440) is configured to exhibit nonlinear behavior in response to forces applied by the elongate movement members. The nonlinear behavior may be based on stiffness and compressibility of the scope (440), as well as variability in slack or stiffness between different elongate movement members.

In some versions, the scope (440) includes at least one sensor that is configured to generate and/or send sensor position data to another device. The sensor position data can indicate a position and/or orientation of the scope (440) (e.g., the distal end (442) thereof) and/or may be used to determine/infer a position/orientation of the scope (440). For example, a sensor (sometimes referred to as a “position sensor”) may include an electromagnetic (EM) sensor with a coil of conductive material or other form of an antenna. In some versions, the position sensor is positioned on the distal end (442) of scope (440), while in other embodiments the sensor is positioned at another location on scope (440).

As shown in FIG. 3, EM field generator (18) is configured to broadcast an alternating EM field 90 that is detected by the EM position sensor of scope (440). The alternating magnetic field (MF) induces small currents in coils of the EM position sensor, which may be analyzed to determine a distance and/or angle/orientation between the EM position sensor and the EM field generator (18). It should be understood that scope (440) may include other types of sensors, such as a shape sensing fiber, accelerometer(s), gyroscope(s), satellite-based positioning sensor(s) (e.g., global positioning system (GPS) sensors), radio-frequency transceiver(s), and so on. In the present example, the EM position sensor of scope (440) provides sensor data to control system (50), which is then used to determine a position and/or an orientation of scope (440).

In some variations, any of the features and aspects described above may be constructed and operable in accordance with at least some of the teachings of U.S. Pat. No. 11,737,663, entitled “Target Anatomical Feature Localization,” issued Aug. 29, 2023, the disclosure of which is incorporated by reference herein, in its entirety; U.S. Pub. No. 2021/0369384, entitled “Stuck Instrument Management,” published Dec. 2, 2021, the disclosure of which is incorporated by reference herein, in its entirety; U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety; and/or U.S. Pub. No. 2022/0096183, entitled “Haptic Feedback for Aligning Robotic Arms,” published Mar. 31, 2022, the disclosure of which is incorporated by reference herein, in its entirety.

### II. Example of Integration of Force Isolation Elements into Steerable Elongate Member

As noted above, it may be desirable to provide an elongate member (e.g., a shaft of scope (440) that has one or more articulating sections, which may allow the elongate member to be actively steered along tortuous anatomy, to provide a movable field of view from a camera at the distal end of the elongate member, to promote access to a targeted anatomical structure with another instrument that is slidably disposed in the elongate member, and/or for other purposes. To the extent that pull-wires and/or other tensioning elements are used to achieve such articulation, it may be further desirable to prevent longitudinal compression from occurring in one or more regions of the elongate member when the elongate member is formed of a material that is otherwise longitudinally compressible. Similarly, it may be desirable to prevent longitudinal extension from occurring in one or more regions of the elongate member when the elongate member is formed of a material that is otherwise longitudinally extensible. Otherwise, undesired longitudinal extension or compression within the elongate member may result in undesired articulation of the elongate member. In scenarios where articulation drive elements (e.g., tendons) might tend to cause undesired longitudinal extension or compression within the elongate member in a first longitudinal region while intentionally driving articulation in a second longitudinal region, it may be beneficial to include force isolation elements that prevent such undesired longitudinal extension or compression within the elongate member in the first longitudinal region.

In some cases, a certain section (e.g., proximal section) of an elongate member body may be formed of a non-compressible/non-extendable material (e.g., steel tubing, etc.); while another section (e.g., distal section) of the elongate member body is formed of a compressible/extendible material (e.g., polymer, braiding, etc.). However, using such different materials may complicate the process of manufacturing the elongate member, may add cost, and/or may have other undesirable consequences. It may therefore be desirable to provide an elongate member that has a body formed of a material that is substantially uniform along the length of the body; while also providing degrees of compressibility/extensibility that vary along the length of the body. It may also be desirable to provide an elongate member with articulation control elements (e.g., tendons) that do not intrude on a working channel of the elongate member or otherwise adversely affect the cross-sectional efficiency of the elongate member.

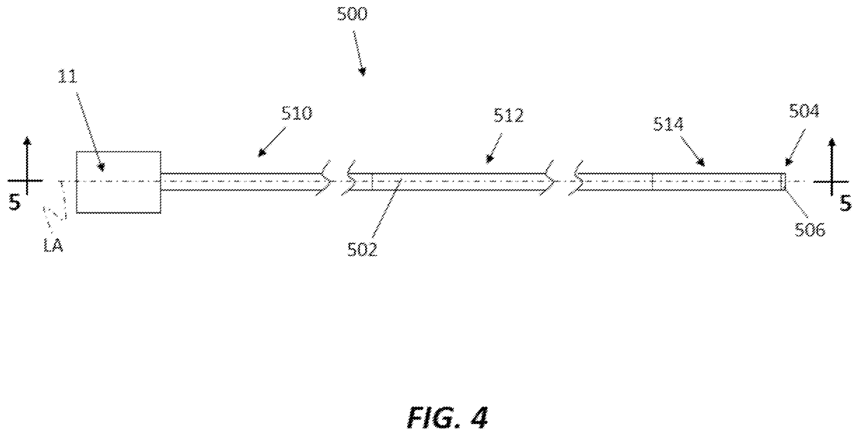

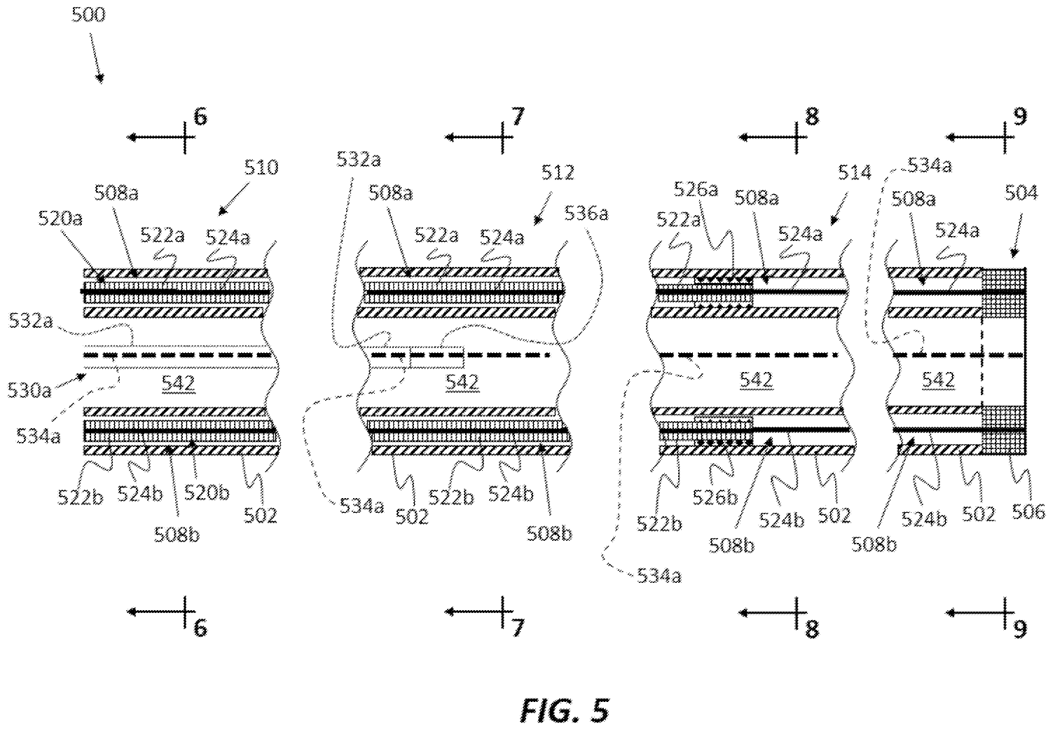

FIGS. 4-5 show an example of an articulating elongate member (500) that may provide one or more of the beneficial features and functionalities referred to above. Articulating elongate member (500) that may be used with robotic surgical system (10). By way of example only, elongate member (500) may represent a variation of scope (40), access sheath (90), or scope (440). Alternatively, elongate member (500) may take the form of a catheter and/or any other suitable kind of elongate instrument. Elongate member (500) of the present example includes a body (502) having a proximal portion (510), a medial portion (512), and a distal portion (514). In the present example, medial portion (512) and distal portion (514) are each operable to articulate, such that distal end (504) of elongate member (500) may be deflected laterally away from and toward a central longitudinal axis (LA) (e.g., defined by proximal portion (510)). In some versions, elongate member (500) is operable to articulate at only one ore at more than two different regions along the length of elongate member (500). For instance, distal portion (514) may include one or more articulation sections, medial portion (512) may include one or more articulation sections, and/or proximal portion (510) may include one or more articulation sections.

Proximal portion (510) is coupled with instrument coupling (11) of robotic surgical system (10), such that robotic surgical system (10) is operable to drive elongate member (500) via instrument coupling (11). By way of example only, robotic surgical system (10) may be operable to drive translation along the central longitudinal axis (LA), rotation (e.g., spinning about the central longitudinal axis (LA)), articulation, and/or other forms of movement of/by elongate member (500).

Distal end (504) of the present example may include one or more openings through which one or more additional instruments may exit into a surgical space or other anatomical region within a patient. Distal end (504) may also include one or more imaging devices, such as an imaging device (448), that may take the form of one or more cameras, one or more optical fibers with corresponding lenses, etc. Distal end (504) may also include one or more illuminating elements, such as one or more integral light-emitting diodes, one or more lenses optically coupled with corresponding optical fibers, etc. In some versions, distal end (504) includes an end effector that is operable to perform one or more operations on tissue, such as grasping, cutting, suturing, sealing (e.g., via RF energy or ultrasonic energy), stapling, etc. In the present example, distal end (504) includes a control ring (504) that is fixedly secured to body (502). Control ring (504) is configured to provide a distal anchoring point for tendons (524, 534), as will be described in greater detail below. To the extent that other components are positioned at distal end (504) (e.g., an imaging device, illuminating elements, an end effector, etc., such additional components may be positioned distal to control ring (504).

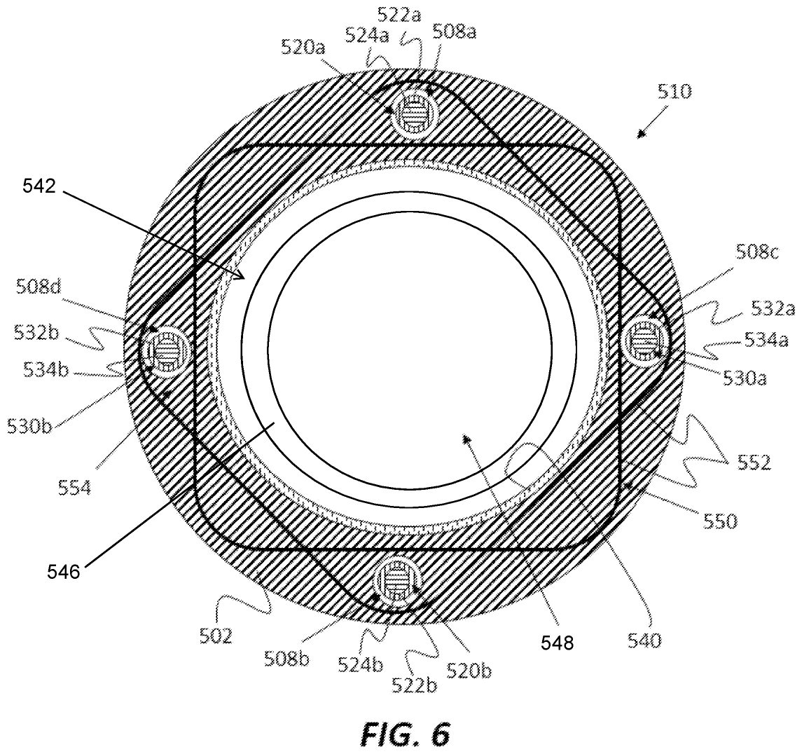

As shown in FIG. 5-8, body (502) of the present example defines an inner lumen (542) and an angularly spaced array of tendon assembly lumens (508). Inner lumen (542) is configured to receive other components. In the present example, inner lumen receives a working channel (546), which may comprise a braided shaft and/or any other suitable components. Working channel (546) defines a lumen (548). Working channel (546) is shown in FIGS. 6-8 but omitted from FIG. 5 for clarity. In some versions, lumen (548) slidably receives other instruments. By way of example only, basket (35) and basketing sheath (37) of basketing device (30) may be advanced distally via lumen (548) of working channel (546). By way of further example only, a laser fiber or other instrument may be disposed in lumen (548) of working channel (546). Alternatively, fluids (e.g., liquid, suction, etc.) may be communicated via lumen (548) of working channel (546). Inner lumen (542) and working channel (546) may extend all the way to distal end (504), where inner lumen (542) may terminate in a distal opening allowing an instrument that is disposed in working channel (546) to exit distally from elongate member (500). In the present example, inner lumen (542) includes a liner (540) that is configured to reduce friction and thereby promote slidability through inner lumen (542). By way of example only, liner (540) may include polytetrafluoroethylene (PTFE), polyimide, and/or any other suitable kind(s) of material(s). While not shown, other features such as electrical wires, optical fibers, flex circuits, etc., may extend along at least part of the length of inner lumen (542), external to working channel (546).

In the present example, tendon assembly lumens (508) are positioned approximately 90 degrees apart from each other about the central longitudinal axis (LA). In the views shown in FIGS. 6-8, tendon lumen (508a) is in the 12 o'clock position, tendon lumen (508b) is in the 6 o'clock position, tendon lumen (508c) is in the 3 o'clock position, and tendon lumen (508d) is in the 9 o'clock position. Tendon lumens (508) extend along the entire length of body (502) in this example. Each tendon assembly lumen (508) contains a corresponding tendon assembly (520, 530). Specifically, tendon assembly (520a) is disposed in tendon lumen (508a) at the 12 o'clock position, tendon assembly (520b) is disposed in tendon lumen (508b) at the 6 o'clock position, tendon assembly (530a) is disposed in tendon lumen (508c) at the 3 o'clock position, and tendon assembly (530b) is disposed in tendon lumen (508d) at the 9 o'clock position. It should be understood that the number of tendon assembly lumens (508) described above, and the angular positioning of tendon assembly lumens (508) described above, is just an illustrative example. Other variations may have any other suitable number of tendon assembly lumens (508), at any other suitable angular positions.

Each tendon assembly (520, 530) includes a housing (522, 532) and a tendon (524, 534) slidably disposed in housing (522, 532). Housing (522, 532) is configured to bend laterally away from the central longitudinal axis (LA); yet not compress longitudinally. By way of example only, housing (522, 532) may be configured as a coil pipe formed of round or square steel wire. As another example, housing (522, 532) may include several wires wrapped in multiple adjacent helixes. As yet another example, housing (522, 532) may comprise a tubular structure formed of steel, high strength plastic, and/or any other suitable material(s), including combinations thereof. In some variations, housing (522, 532) comprises a stainless steel hypotube. Alternatively, housing (522, 532) may take any other suitable form. In some variations, housing (522, 532) includes a low-friction (e.g., polytetrafluoroethylene (PTFE), polyimide, etc.) lining within the lumen in which tendon (524, 534) is disposed. Housing (522, 532) may also include a lubricious interface on the exterior of housing (522, 532), thereby facilitating sliding of housing (522, 532) within tendon assembly lumen (508).

Similarly, instead of using laser cut hypotubes to form housings (522, 532), housings (522, 532) may comprise coil pipes. Such coil pipes may be stretched longitudinally along the distal regions of the coil pipes to plastically deform the coils to effectively open up the coils along the distal regions of the coil pipes. Such plastically deformed distal regions may be positioned along the articulating distal region of elongate member (500), similar to the laser cut regions of the hypotubes described above. Such plastically deformed distal regions may provide the desired amount of compression along distal portion (514) of elongate member (500).

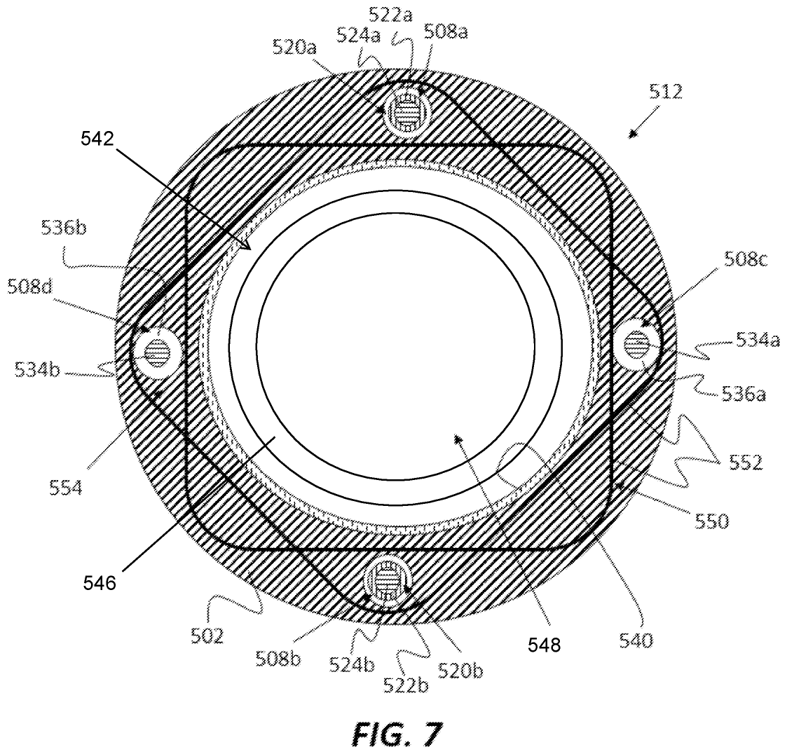

As shown in FIGS. 5 and 6, housings (522, 532) all extend along the full length of proximal portion (510). As shown in FIGS. 5 and 7, housings (522) continue to extend through medial portion (512) while housings (532) distally terminate in medial portion (512). An adhesion is provided within tendon assembly lumens (508) to fixedly secure the distal portions of housings (532) relative to body (502) within medial portion (512). By way of example only, this adhesion may be provided via a separate adhesive that is applied within tendon assembly lumens (508). By way of further example only, this adhesion may be provided by reflowing material forming body (502), reflowing material forming housing (532), and/or providing some other form of thermal bonding process. Alternatively, an adhesion may be formed between distal portions of housings (532) and body (502) in any other suitable fashion. It should also be understood that the adhesion may be isolated to just the distal ends of housings (532); or may extend proximally along housings (532) from the distal ends to any suitable extent.

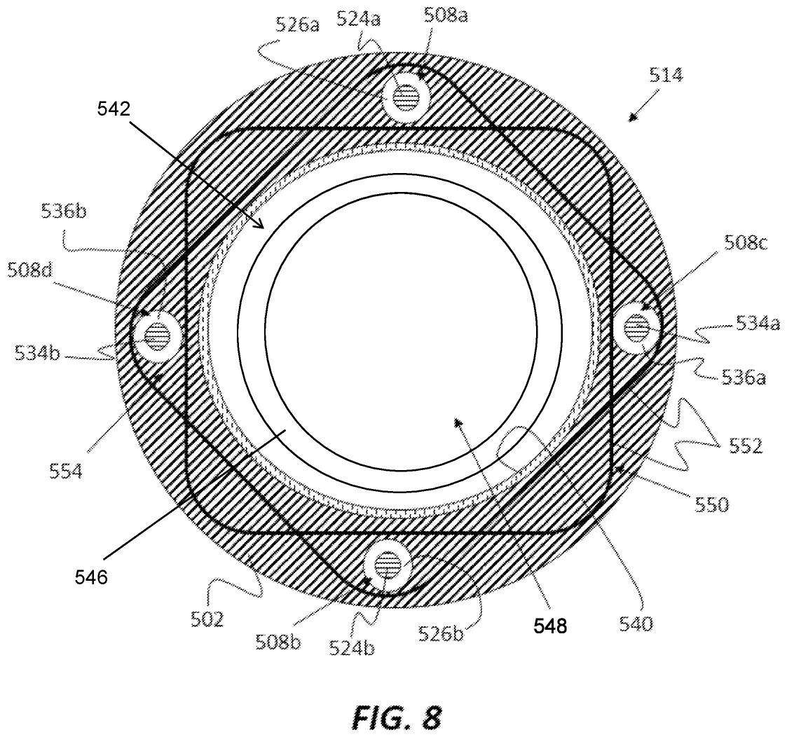

As shown in FIGS. 5 and 8, housings (522) distally terminate in distal portion (514). An adhesion (526) is provided within tendon assembly lumens (508) to fixedly secure the distal portions of housings (522) relative to body (502) within distal portion (514). By way of example only, this adhesion (526) may be provided via a separate adhesive that is applied within tendon assembly lumens (508). By way of further example only, this adhesion (526) may be provided by reflowing material forming body (502), reflowing material forming housing (522), and/or providing some other form of thermal bonding process. Alternatively, an adhesion (526) may be formed between distal portions of housings (522) and body (502) in any other suitable fashion. It should also be understood that adhesion (526) may be isolated to just the distal ends of housings (522); or may extend proximally along housings (522) from the distal ends to any suitable extent.

In some variations, housings (532) distally terminate in distal portion (514), in addition to housings (522) distally terminating in distal portion (514). In some such versions where housings (522, 532) comprise stainless steel hypotubes, the distal regions of housings (522, 532) may be laser cut to create a compression spring (i.e., a spring portion of housing (522, 532)). Housings (532) may be fixedly secured to body (502) and/or braid assembly (550) (e.g., via thermal bonding, etc.), just proximal to distal portion (514). The spring portions of housings (522, 532) extending along distal portion (514) may allow compression as needed for articulation of distal portion (514). The pitch of spring portions of housings (522, 532) may be adjusted as needed to provide variable stiffness/allowable amounts of compression (prior to the spring portions becoming fully stacked). This may allow the articulation shape to be better tuned as desired. In addition, this arrangement may allow that braid assembly (550) to have a fixed lumen size over its entire length without worry of excessive clearance/deformability in the articulating section (between inner lumen 542 and tendon assemblies (520, 530)).

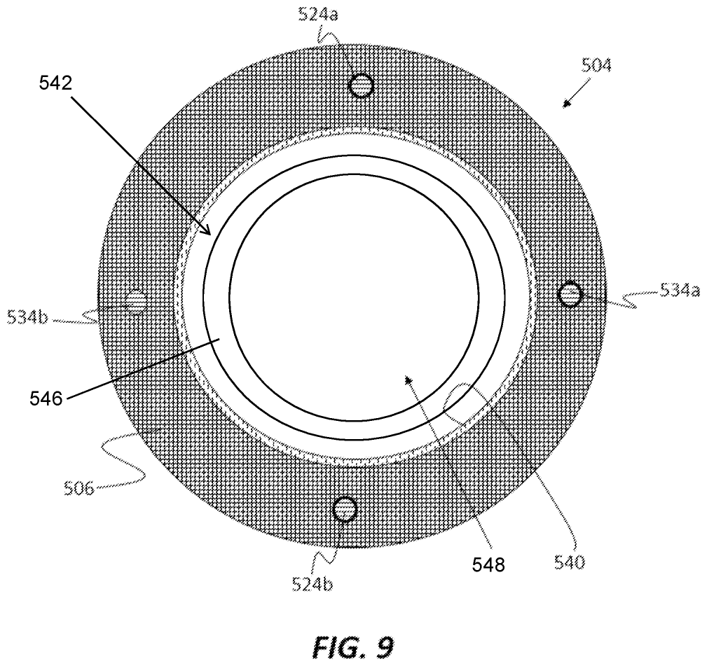

Returning to the present example, as shown in FIGS. 5-9, each tendon (524, 534) extends along the full length of body (502), with distal ends of tendons (524, 534) being fixedly secured at control ring (506), and with control ring (506) being fixedly secured at the distal end of body (502). By way of example only, each tendon (524, 534) may comprise a pull wire, a drive band, a single-strand cable, a multi-strand cable, one or more metals, one or more fibers, and/or any other suitable component that is operable to communicate a pulling force along the length of elongate member (500), to thereby provide articulation of elongate member (500), without substantially stretching. Such tendons (524, 534) may also be coupled with instrument coupling (11) of robotic surgical system (10), such that robotic surgical system (10) is operable to drive tendons (524, 534) via instrument coupling (11).

As described above, housings (532) distally terminate in medial region (512); while housings (522) distally terminate in distal region (514). As also described above, housings (532) are configured to not compress longitudinally. Thus, when tension is applied to either of tendons (524), the region of distal portion (514) that is distal to the distal end of the corresponding housing (522) will articulate; while housing (522) will provide isolation against compressive forces along the entire length of body (502) that is proximal to the distal end of housing (522). In other words, the region of body (502) that is proximal to the distal end of housing (522) will not deform in response to tension being applied to the corresponding tendon (524). Similarly, when tension is applied to either of tendons (534), distal portion (514) and the region of medial portion (512) that is distal to the distal end of the corresponding housing (532) will articulate; while housing (532) will provide isolation against compressive forces along the entire length of body (502) that is proximal to the distal end of housing (532). In other words, the region of body (502) that is proximal to the distal end of housing (532) will not deform in response to tension being applied to the corresponding tendon (534). It should be understood from the foregoing that tendon assemblies (520, 530) may be configured and operable like Bowden cable assemblies.

In some variations, housings (522, 532) do not distally terminate in the positions described above and shown in FIG. 5. Some such variations may include an additional housing structure that is used just for adhesive termination like a plug made of a similar material as described above (e.g., metal or polymer, composite, etc.). For instance, a short section made of housing (522, 532) may be placed in tendon assembly lumen (508) and be bonded thereto. This may thus form a subassembly. The remaining length of housing (522, 532) may abut this subassembly and would not need to be bonded to tendon assembly lumen (508).

In the present example, tendon assemblies (520, 530) extend along respective straight paths that are parallel to the central longitudinal axis (LA), along the full length of elongate member (500). In some other versions, tendon assemblies (520, 530) may extend along helical paths along at least part of the length of elongate member (500). As shown in FIGS. 6-8, tendons (524) are positioned along a first plane (i.e., a vertical plane in the views of FIGS. 6-8) that extends along the central longitudinal axis (LA); while tendons (534) are positioned along a first plane (i.e., a horizontal plane in the views of FIGS. 6-8) that extends along the central longitudinal axis (LA)). Tendons (524) are thus operable to drive articulation along a plane that is orthogonal to the plane along which tendons (534) are operable to drive articulation. Tendons (532, 534) may be driven independently in the present example. Thus, distal portion (514) may be articulated in a first direction while medial portion (512) is articulated in a second direction. It should be understood that any other suitable number of tendon assemblies may be provided in any other suitable angular positioning(s) about the central longitudinal axis (LA); and that any alternative tendon assembly may have a housing that distally terminates with adhesion at any suitable position along the length of body (502). Such angular positioning variations, and housing distal end adhesion position variations, may be selected and combined to provide a certain desired articulation profile.

In some versions, the proximal ends of housings (522, 532) are fixedly secured relative to instrument coupling (11) (or relative to a handle or other structure); but not relative to the proximal end of body (502). In some such versions, the entire length of each housing (522, 532) that is proximal to adhesion (526, 536) may slide longitudinally relative to body (502). In other words, the proximal ends each housing (522, 532) may be configured to translate longitudinally relative to the region of body (502) that is proximal to adhesion (526, 536). In some such versions, such slidability of the proximal ends of housings (522, 532) relative to body (502) may allow proximal portion (510) of elongate member (500) to bend laterally (e.g., to traverse tortuous anatomical structures) without causing undesired articulation of medial portion (512) or distal portion (514) of elongate member (500). To promote such slidability, the exterior regions of housings (522, 532) and/or the interior regions of tendon assembly lumens (508) may include a lubricious material (e.g., polytetrafluoroethylene, etc.).

As also shown in FIGS. 6-8, elongate member (500) of the present example includes a braid assembly (550). Braid assembly (550) is wrapped about liner (540) and is embedded within body (502). Braid assembly (550) includes a plurality of wire strands (552) that are wrapped to form an elongated braided structure that extends along the length of liner (540) and body (502). Tendon assemblies (520, 530) effectively intertwined within braid assembly (550), such that each tendon assembly (520, 530) is disposed in a space (554) defined by wire strands (552). As described in greater detail below, tendon assemblies (520, 530) may be effectively captured between braid assembly (550) and liner (540) in some versions; while in other versions, tendon assemblies (520, 530) may be effectively integrated into braid assembly (550) by being interwoven with strands (552).

Body (502) of the present example is formed about the exterior of braid assembly (550). By way of example only, body (502) may be formed about the exterior of braid assembly (550) through a reflow process and/or through any other suitable process. Body (502) may comprise a reflow material such as polyether block amide (PEBA) and/or any other suitable kind(s) of material(s). At least some of the material used to form the region of body (502) outside of braid assembly (550) may also reach the region between braid assembly (550) and liner (540), as shown in FIGS. 6-8. In scenarios where any of tendon assemblies (520, 530) would otherwise tend to exert outwardly/outwardly directed forces on body (520) (e.g., during bending of elongate member (500), particularly during driven articulation), braid assembly (550) may effectively absorb such forces and thereby shield body (502) from any damage that might otherwise be caused to body (502) by tendon assemblies (520, 530). Braid assembly (550) thus provides structural reinforcement to body (502). However, braid assembly (550) still allows body (502) to flexibly deflect laterally, such as during traversal of tortuous anatomical structures and/or during actively driven articulation.

### III. Example of Apparatus and Method to Manufacture Steerable Elongate Member with Integral Force Isolation Elements

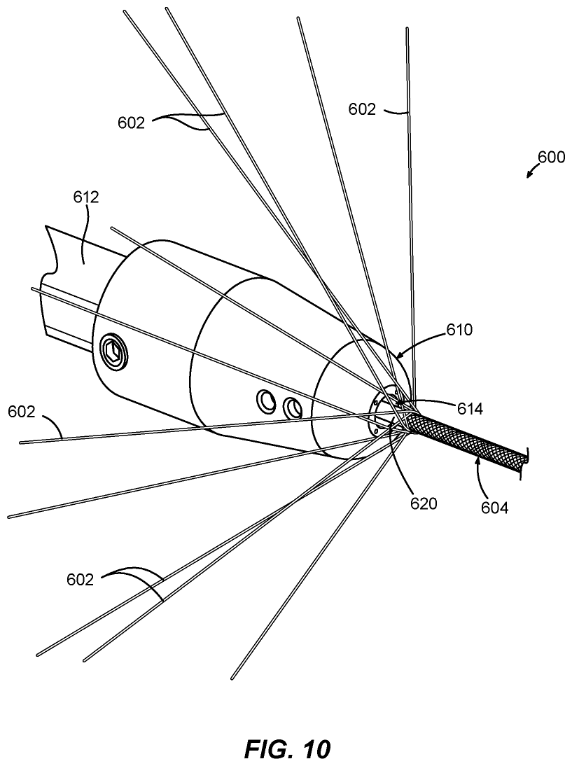

FIG. 10 depicts an example of an apparatus (600) that may be used in a process to manufacture at least part of an elongate member that includes braided structures. In particular, FIG. 10 depicts a head (610) at the distal end of a shaft (612), with a mandrel (620) disposed in a central opening (614) defined by head (610). Head (610) has a rounded, generally conical shape in this example. The proximal end of shaft (612) may be coupled with a motor (not shown), which may be operable to rotate shaft (612) and head (610) about the central longitudinal axis shared by shaft (612) and mandrel (620). An actuator (not shown) may be coupled with mandrel (620) to drive mandrel (620) longitudinally relative to shaft (612). Alternatively, an actuator may be coupled with shaft (612) to drive shaft (612) and head (610) longitudinally relative to mandrel (620).

Regardless of whether head (610) rotates relative to mandrel (620) during longitudinal translation of mandrel (620) relative to head (610) (or during longitudinal translation of head (610) relative to mandrel (620)), a plurality of braid strands (602) may be wrapped about mandrel (620) (or onto structure(s) predisposed on mandrel (620)) to thereby form a braid about mandrel (620) (and about any structure(s) predisposed on mandrel (620)). Such braid-wrapping of strands (602) may be carried out through a coordinated movement of various spools, etc., from which strands (602) are fed, using known components and techniques. The rounded, generally conical shape of head (610) may support strands (602) and thereby assist in guiding strands (602) into place along mandrel (620) (or onto structure(s) predisposed on mandrel (620)) as strands (602) are being wrapped to form the braid (604).

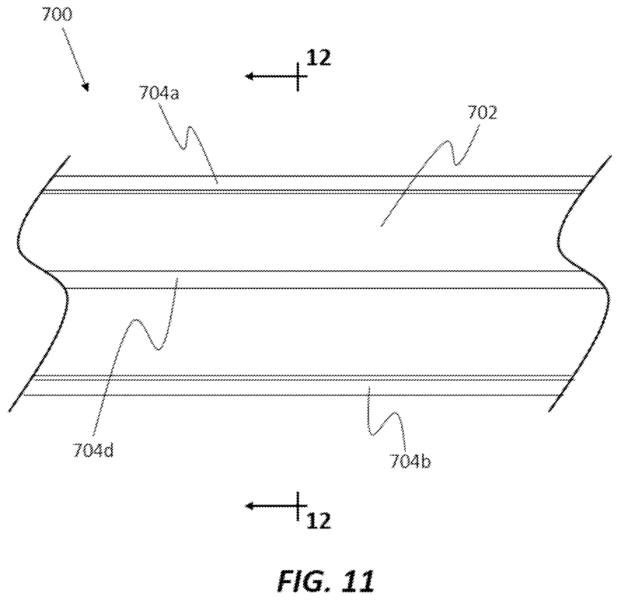

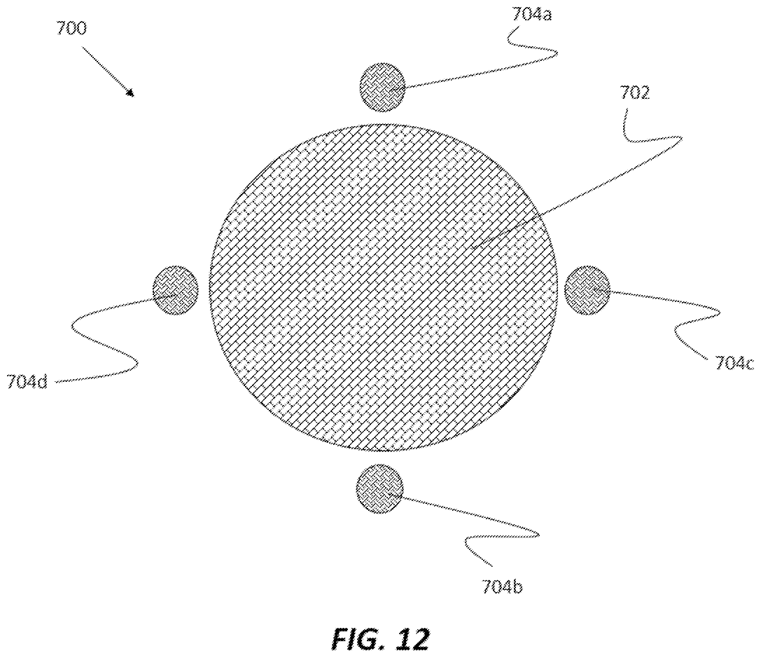

As noted above, it may be desirable to form an elongate member (500) in which tendon assemblies (520, 530) effectively intertwined within braid assembly (550), such that each tendon assembly (520, 530) is disposed in a space (554) defined by wire strands (552). FIGS. 11-12 show an example of a mandrel set (700) that may be employed in a variation of apparatus (600) during a process of manufacturing elongate member (500). Mandrel set (700) of this example includes a central mandrel (702) and an array of outer mandrels (704) angularly spaced about central mandrel (702). Central mandrel (702) may effectively serve as mandrel (620) described above, such that central mandrel (702) is longitudinally translatable through central opening (614) of head (610). The diameter of central mandrel (702) corresponds to the diameter of inner lumen (542).

In some versions, outer mandrels (704) are fixedly predisposed in the positions shown in FIG. 12. In some such versions, outer mandrels (704) fit through central opening of head (610) with central mandrel (702). This may allow outer mandrels (704) to be effectively captured within braid assembly (550), against liner (540), without necessarily being interwoven among strands (552). In some other versions, outer mandrels (704) are laterally flexible and are fed along mandrel (702) with strands (552), as guided by head (610). This may promote interweaving of mandrels (704) among strands (552), such that mandrels (704) cooperate with strands (552) to be effectively integrated into braid assembly (550).

The position and diameter of outer mandrel (704a) ultimately corresponds to the position and diameter of tendon assembly lumen (508a). The position and diameter of outer mandrel (704b) ultimately corresponds to the position and diameter of tendon assembly lumen (508b). The position and diameter of outer mandrel (704c) ultimately corresponds to the position and diameter of tendon assembly lumen (508c). The position and diameter of outer mandrel (704d) ultimately corresponds to the position and diameter of tendon assembly lumen (508d).

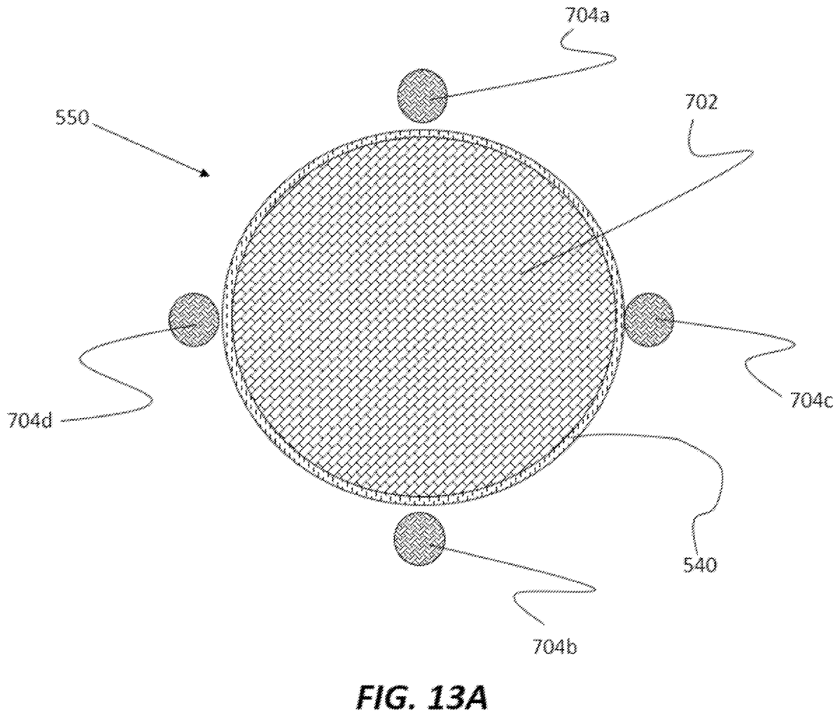

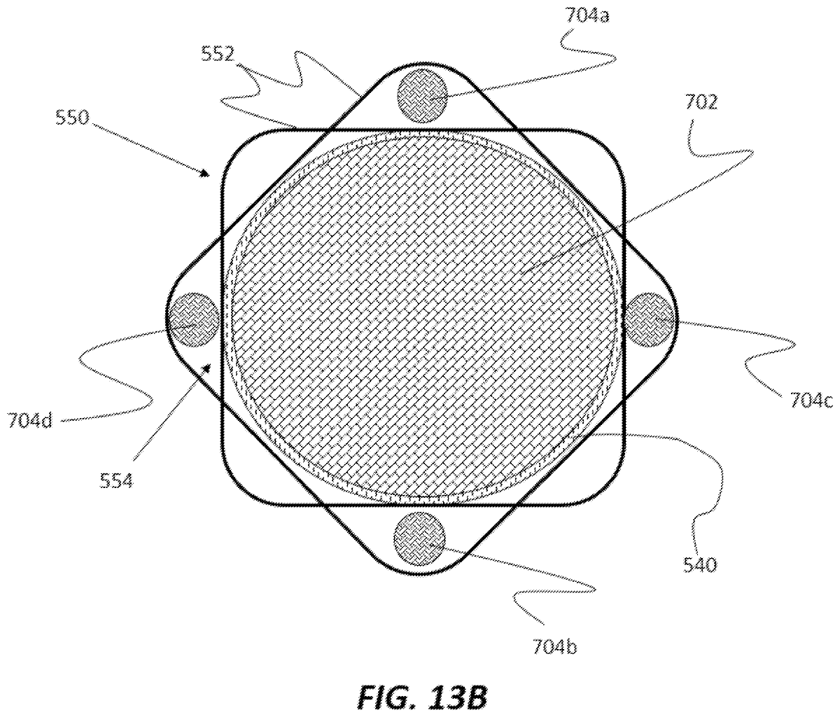

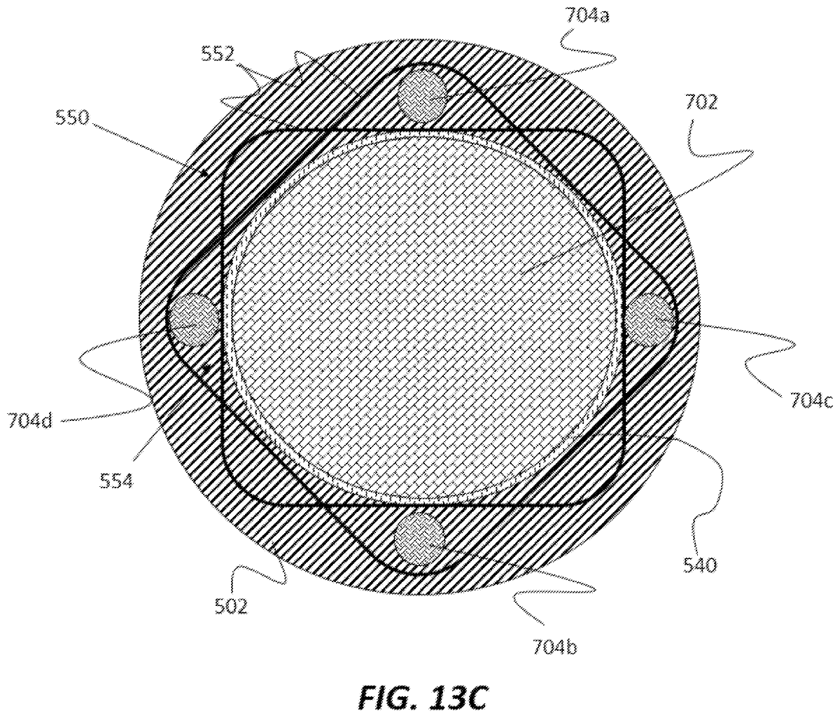

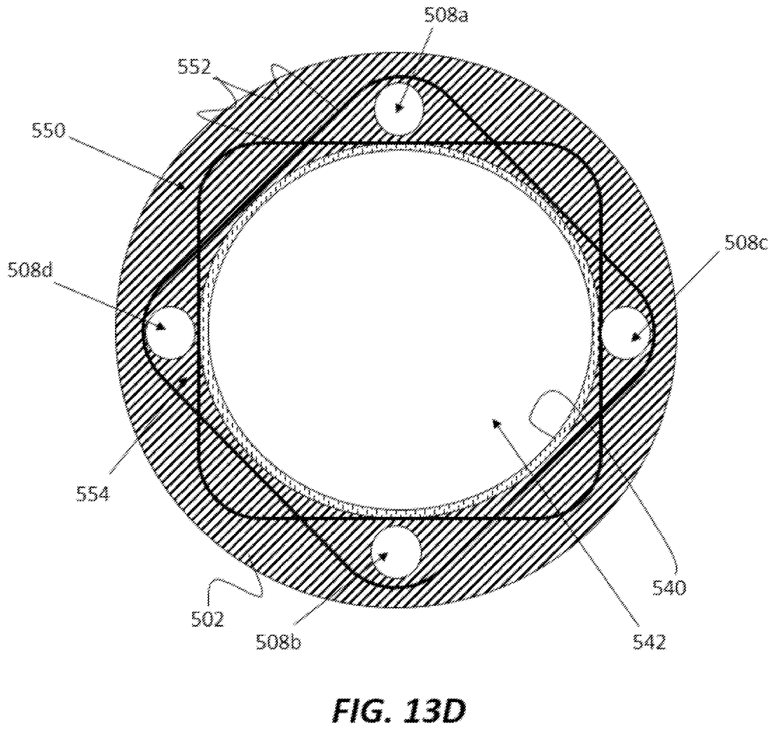

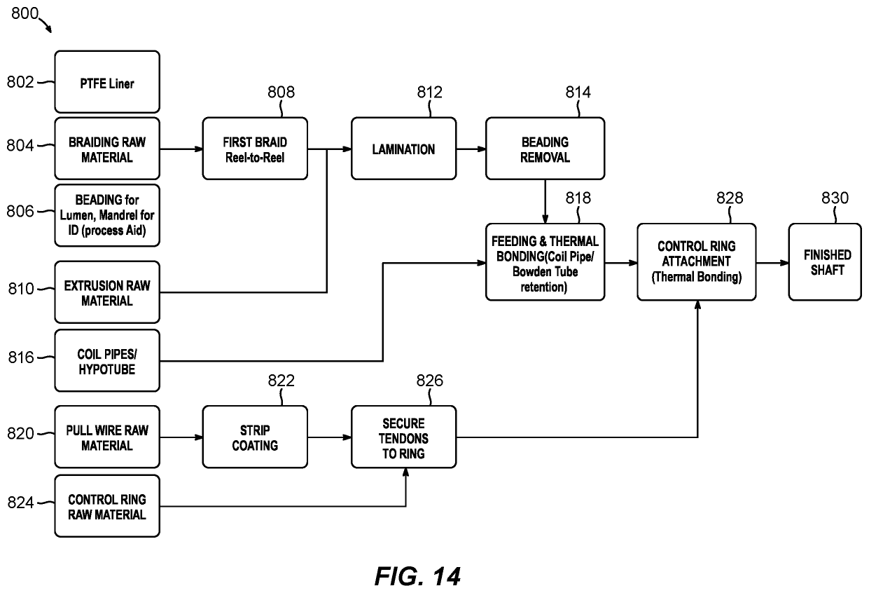

FIGS. 13A-13D and 14 show examples of materials and steps that may be used to manufacture elongate member (500) using a version of apparatus (600) that includes mandrel set (700). In particular, FIG. 14 shows a set (800) of components that include a low-friction material (802) (e.g., polytetrafluorethylene, polyimide, etc.) to form liner (540); a material (804) (e.g., stainless steel, aramid fiber, etc.) to form strands (552) that will ultimately form braid assembly (550); one or more materials (806) (e.g., steel and/or polymer) used to form mandrels (702, 704); a material (810) (e.g., polyether block amide (PEBA), etc.) to form body (502); a material (816) (e.g., steel wire coil pipe, steel hypotube, a non-compressible yet laterally flexible polymer structure, etc.) to form housings (522, 532)); a material (820) (e.g., stainless steel, aramid fiber, tungsten, carbon fiber, etc.) to form tendons (524, 534); and a material (824) (e.g., stainless steel, a rigid polymer, etc.) to form control ring (506).

At the beginning of the manufacturing process, a lubricant may be applied to at least outer mandrels (704); and in some cases, to central mandrel (702). Then, liner (540) is positioned about central mandrel (702), as shown in FIG. 13A. Strands (552) are then wrapped with (or about) about outer mandrels (704) and central mandrel (702) while liner (540) is disposed on central mandrel, such that braid assembly (550) is formed about liner (540) as shown in FIG. 13B. This braid forming process is also represented in block (808) of FIG. 14. As noted above, in some versions of this braid forming process, outer mandrels (704) are guided by head (610) and fed/wrapped with strands (552) around liner (540) and central mandrel (702). In such versions, outer mandrels (704) are interwoven with strands (552) to be effectively integrated into braid assembly (550).

Regardless of whether outer mandrels (704) are effectively captured between braid assembly (550) and liner (540) or cooperate with strands (552) to be effectively integrated into braid assembly (550), the material (810) that is used to form body (502) may then applied about braid assembly (550) and mandrel set (700), as represented by block (812) of FIG. 14. As noted above, this process may include a reflow process, a molding process, an extrusion process, and/or any other suitable process to form body (502). In any case, the resulting formation may appear as shown in FIG. 13C. It should be understood that braid assembly (550) is coextensive with body (502) in the present example, such that braid assembly (550) extends along the full length of body (502) and vice-versa.

With body (502) formed, mandrel assembly (700) is removed, as represented by block (814) of FIG. 14. As noted above, a lubricant may be applied to at least outer mandrels (704), such that the lubricant may facilitate removal of mandrels (704) from body (502) and braid assembly (550). In either case, the resulting formation may appear as shown in FIG. 13D.

As shown in FIG. 13D, tendon assembly lumens (508) are formed at this stage, ready for receipt of corresponding tendon assemblies (520, 530). The material (816) forming housings (522, 532) is then inserted into the corresponding tendon assembly lumens (508) and secured at the appropriate longitudinal positions via adhesions (526, 536), as represented by block (818) of FIG. 14. The inner diameters of tendon assembly lumens (508) may be sufficiently larger than the outer diameters of housings (522, 532) such that lubricant may not be necessary to facilitate insertion of housings (522, 532) in corresponding tendon assembly lumens (508). By way of example only, the inner diameters of tendon assembly lumens (508) may be at least approximately 0.002 inches greater than the outer diameters of housings (522, 532).

In versions where adhesions (526, 536) are formed through thermal bonding, heat may be focused on the longitudinal regions of body (502) corresponding to the distal portions of housings (522, 532) and/or anywhere else where it may be desirable to form adhesions (526, 536) along the length of body (502). To the extent that some of the heat reaches a region of lumens (508) that do not contain housings (522, 532), the heat may tend to slightly reduce the inner diameter of such lumens (508) without necessarily causing such lumens to effectively collapse upon tendons (524, 534). In some variations, housings (522, 532) include an outer polymer layer (not shown) along the distal region where adhesions (526, 536) will be formed. Such an outer polymer layer may tend to enhance the adhesion strength at adhesions (526, 536).

In the present example, the material (820) forming tendons (524, 534) is predisposed in housings (522, 532), such that the process does not entail an additional step of feeding tendons (524, 534) into corresponding housings (522, 532). However, some variations of the process may include such a feeding step. Another optional step may include stripping a coating off the distal ends of tendons (524, 534), as represented by block (822) of FIG. 14, to promote coupling of tendons (524, 534) with control ring (506) as described below. However, this step may be omitted in some versions (e.g., based on the existing configuration of tendons (524, 534), such as those that lack a coating, etc.). Regardless of whether a stripping step (822) is employed, the distal ends of tendons (524, 534) may be fixedly secured to control ring (506), as represented by block (826) of FIG. 14. By way of example only, this step (826) may include laser welding, soldering, adhesive bonding, or any other suitable technique (and/or fastening components).

With tendon assemblies (520, 530) secured in corresponding tendon assembly lumens (508), and with distal ends of tendons (524, 534) fixedly secured to control ring (506), control ring (506) is then fixedly secured to the distal end of body (502). This is represented by block (828) of FIG. 14. At this stage, the manufacturing process for elongate member (500) may be deemed complete, as represented by block (830) of FIG. 14. This may be particularly so in cases where elongate member (500) serves as a guide sheath or guide catheter. In some other versions, such as when elongate member (500) serves as a variation of scope (400), one or more imaging devices (e.g., an imaging device (448)), one or more illuminating elements (e.g., one or more integral light-emitting diodes, one or more lenses optically coupled with corresponding optical fibers, etc.), and/or other features may be secured at or near control ring (506). Alternatively, an end effector (e.g., basket (35) or other feature that is operable to perform one or more operations on tissue, such as grasping, cutting, suturing, sealing, stapling, etc.) may be secured at or near control ring (506). Other suitable steps that may be carried out after completing the process shown in FIGS. 13A-13D and 14 may be apparent to those skilled in the art in view of the teachings herein.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) an elongate body the elongate body defining a central longitudinal axis, the elongate body including: (i) a sidewall comprising a flexible material, (ii) a proximal portion, and (iii) a distal portion, the distal portion terminating at a distal end; and (b) a first tendon assembly, the first tendon assembly being operable to drive deflection of a portion of the elongate body away from the central longitudinal axis, the first tendon assembly including: (i) a first tendon housing extending through the sidewall at a first angular position about the central longitudinal axis, the first tendon housing having a distal end secured at a first longitudinal position along the elongate body, and (ii) a first tendon slidably disposed in the first tendon housing, the first tendon having a distal portion extending distally from the distal end of the first tendon housing, the distal portion of the first tendon being fixedly secured relative to the elongate body.

### Example 2

The apparatus of Example 1, the flexible material being one or both of longitudinally extensible or longitudinally compressible.

### Example 3

The apparatus of any of Examples 1 through 2, the flexile material comprising polyether block amide.

### Example 4

The apparatus of any of Examples 1 through 3, the first tendon housing being longitudinally non-compressible.

### Example 5

The apparatus of any of Examples 1 through 4, the first tendon housing comprising a coil pipe.

### Example 6

The apparatus of any of Examples 1 through 5, the first tendon comprising a pull wire.

### Example 7

The apparatus of any of Examples 1 through 6, the first tendon assembly forming a Bowden cable assembly.

### Example 8

The apparatus of any of Examples 1 through 7, the first longitudinal position being proximal to the distal end of the distal portion of the elongate body.

### Example 9

The apparatus of any of Examples 1 through 8, further comprising an anchoring element at the distal end of the distal portion of the elongate body.

### Example 10

The apparatus of Example 9, the distal portion of the first tendon being fixedly secured to the anchoring element.

### Example 11

The apparatus of any of Examples 9 through 10, the anchoring element including a control ring.

### Example 12

The apparatus of any of Examples 1 through 11, the first tendon assembly being operable to drive deflection of the distal portion of the elongate body away from the central longitudinal axis.

### Example 13

The apparatus of any of Examples 1 through 12, the elongate body further defining an inner lumen.

### Example 14

The apparatus of Example 13, the inner lumen being coaxial with the central longitudinal axis.

### Example 15

The apparatus of any of Examples 13 through 14, further comprising a liner in the inner lumen, such that the liner is radially interposed between the sidewall and the inner lumen.

### Example 16

The apparatus of Example 15, the liner comprising polytetrafluoroethylene or polyimide.

### Example 17

The apparatus of any of Examples 1 through 16, further comprising a braid assembly.

### Example 18

The apparatus of Example 17, the braid assembly being positioned within the sidewall.

### Example 19

The apparatus of any of Examples 17 through 18, the braid assembly comprising strands, the first tendon assembly being interwoven with the strands such that the first tendon assembly is integrated into the braid assembly.

### Example 20

The apparatus of any of Examples 1 through 19, further comprising a second tendon assembly, the second tendon assembly being operable to drive deflection of a portion of the elongate body away from the central longitudinal axis, the second tendon assembly including: (i) a second tendon housing extending through the sidewall at a second angular position about the central longitudinal axis, the second tendon housing having a distal end secured to the elongate body, and (ii) a second tendon slidably disposed in the second tendon housing, the second tendon having a distal portion extending distally from the distal end of the second tendon housing, the distal portion of the second tendon being fixedly secured relative to the elongate body.

### Example 21

The apparatus of Example 20, the second angular position being angularly offset from the first angular position by approximately 90 degrees.

### Example 22

The apparatus of Example 20, the second angular position being angularly offset from the first angular position by approximately 180 degrees.

### Example 23

The apparatus of any of Examples 20 through 22, the distal end of the second tendon housing being secured to the elongate body at a second longitudinal position along the elongate body.

### Example 24

The apparatus of Example 23, the second longitudinal position being proximal to the first longitudinal position.

### Example 25

The apparatus of Example 24, the elongate body further including a medial portion between the proximal portion and the distal portion, the first tendon assembly being operable to drive deflection of the distal portion of the elongate body away from the central longitudinal axis, the second tendon assembly being operable to drive deflection of the medial portion of the elongate body away from the central longitudinal axis.

### Example 26

The apparatus of any of Examples 20 through 25, the first tendon assembly being operable to drive deflection of the distal portion of the elongate body away from the central longitudinal axis along a first articulation plane, the second tendon assembly being operable to drive deflection of the distal portion of the elongate body away from the central longitudinal axis along a second articulation plane.

### Example 27

The apparatus of Example 26, the second articulation plane being orthogonal to the first articulation plane.

### Example 28

An apparatus comprising: (a) an elongate body the elongate body defining a central longitudinal axis, the elongate body including: (i) a sidewall comprising a flexible material, (ii) a proximal portion, and (iii) a distal portion, the distal portion terminating at a distal end; (b) a tendon assembly extending through the sidewall, the tendon assembly being operable to drive deflection of a portion of the elongate body away from the central longitudinal axis; and (c) a braid assembly positioned within the sidewall, the braid assembly comprising strands, the tendon assembly being interwoven with the strands such that the tendon assembly is integrated into the braid assembly.

### Example 29

The apparatus of Example 28, the tendon assembly including: (i) a tendon housing extending through the sidewall, the tendon housing having a distal end secured at a longitudinal position along the elongate body, and (ii) a tendon slidably disposed in the tendon housing, the tendon having a distal portion extending distally from the distal end of the tendon housing, the distal portion of the tendon being fixedly secured relative to the elongate body.

### Example 30

An apparatus comprising: (a) an elongate body the elongate body defining a central longitudinal axis, the elongate body including: (i) a sidewall comprising a flexible material, (ii) a proximal portion, (iii) a distal portion, the distal portion terminating at a distal end, and (iv) a medial portion between the proximal portion and the distal portion; (b) a first Bowden cable assembly extending through the sidewall, the first Bowden cable assembly being operable to drive deflection of the distal portion of the elongate body away from the central longitudinal axis; and (c) a second Bowden cable assembly extending through the sidewall, the second Bowden cable assembly being operable to drive deflection of the medial portion of the elongate body away from the central longitudinal axis.

### Example 31

The apparatus of Example 30, the first Bowden cable assembly including a first housing having a distal end secured at a first longitudinal position along the elongate body, the second Bowden cable assembly including a second housing having a distal end secured at a second longitudinal position along the elongate body, the second longitudinal position being proximal to the first longitudinal position.

### Example 32

An apparatus comprising: (a) an elongate body the elongate body defining a central longitudinal axis, the elongate body including: (i) a sidewall comprising a flexible material, (ii) a proximal portion, (iii) a distal portion, the distal portion terminating at a distal end, and (iv) a medial portion between the proximal portion and the distal portion; (b) a first Bowden cable assembly extending through the sidewall, the first Bowden cable assembly being operable to drive deflection of a portion of the elongate body away from the central longitudinal axis, the first Bowden cable assembly being positioned at a first angular position about the central longitudinal axis; and (c) a second Bowden cable assembly extending through the sidewall, the second Bowden cable assembly being operable to drive deflection of a portion of the elongate body away from the central longitudinal axis, the second Bowden cable assembly being positioned at a second angular position about the central longitudinal axis.

### Example 33

The apparatus of Example 32, the first Bowden cable assembly being operable to drive deflection of the distal portion of the elongate body away from the central longitudinal axis in a first direction, the second Bowden cable assembly being operable to drive deflection of the distal portion of the elongate body away from the central longitudinal axis in a second direction.

### Example 34

A method comprising: (a) translating a central mandrel relative to a head; (b) positioning a plurality of outer mandrels about the central mandrel; and (c) wrapping a plurality of strands about the central mandrel and the outer mandrels to form a braid assembly while the central mandrel translates relative to the head.

### Example 35

The method of Example 34, further comprising positioning a liner about the central mandrel, such that the braid assembly is formed about the liner.

### Example 36

The method of Example 35, the liner comprising polytetrafluoroethylene or polyimide.

### Example 37

The method of any of Examples 34 through 36, positioning the plurality of outer mandrels about the central lumen including guiding the outer mandrels along the head while the central mandrel translates relative to the head, the plurality of outer mandrels also translating relative to the head while the central mandrel translates relative to the head.

### Example 38

The method of any of Examples 34 through 37, the wrapping the plurality of strands about the central mandrel and the outer mandrels to form a braid assembly further including interweaving the plurality of mandrels with the plurality of strands, thereby incorporating the plurality of strands into the braid assembly.

### Example 39

The method of any of any of Examples 34 through 38, further comprising forming an elongate body about the braid assembly, the central mandrel and the plurality of outer mandrels remaining within the braid assembly while forming the elongate body about the braid assembly.

### Example 40

The method of Example 39, the forming the elongate body including one or more of a reflow process, an overmold process, or an extrusion process.

### Example 41

The method of any of Examples 39 through 40, further comprising removing the central mandrel and the plurality of outer mandrels from the combination of the elongate body and the braid assembly, the removal of the central mandrel forming an inner lumen along the combination of the elongate body and the braid assembly, the removal of the plurality of outer mandrels forming a plurality of tendon assembly lumens.

### Example 42

The method of Example 41, further comprising inserting tendon assemblies in the tendon assembly lumens, each tendon assembly comprising a tendon housing and a tendon.

### Example 43

The method of Example 42, further comprising fixedly securing a distal portion of each tendon housing to the elongate body.

### Example 44

The method of any of Examples 42 through 43, further comprising fixedly securing a distal end of each tendon relative to the elongate body.

### Example 45

The method of Example 44, further comprising: (a) fixedly securing a distal end of each tendon to an anchoring member; and (b) fixedly securing the anchoring member to a distal end of the elongate body.

### V. Miscellaneous

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions described above may be designed to be disposed of after a single use, or they may be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that may penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) an elongate body the elongate body defining a central longitudinal axis, the elongate body including:
(i) a sidewall comprising a flexible material,
(ii) a proximal portion, and
(iii) a distal portion, the distal portion terminating at a distal end; and
(b) a first tendon assembly, the first tendon assembly being operable to drive deflection of a portion of the elongate body away from the central longitudinal axis, the first tendon assembly including:
(i) a first tendon housing extending through the sidewall at a first angular position about the central longitudinal axis, the first tendon housing having a distal end secured at a first longitudinal position along the elongate body, and
(ii) a first tendon slidably disposed in the first tendon housing, the first tendon having a distal portion extending distally from the distal end of the first tendon housing, the distal portion of the first tendon being fixedly secured relative to the elongate body.

2. The apparatus of claim 1, the flexible material being one or both of longitudinally extensible or longitudinally compressible.

3. The apparatus of claim 1, the flexile material comprising polyether block amide.

4. The apparatus of claim 1, the first tendon housing being longitudinally non-compressible.

5. The apparatus of claim 1, the first tendon housing comprising a coil pipe.

6. The apparatus of claim 1, the first tendon comprising a pull wire.

7. The apparatus of claim 1, the first tendon assembly forming a Bowden cable assembly.

8. The apparatus of claim 1, the first longitudinal position being proximal to the distal end of the distal portion of the elongate body.

9. The apparatus of claim 1, further comprising an anchoring element at the distal end of the distal portion of the elongate body.

10. The apparatus of claim 1, the first tendon assembly being operable to drive deflection of the distal portion of the elongate body away from the central longitudinal axis.

11. The apparatus of claim 1, the elongate body further defining an inner lumen.

12. The apparatus of claim 1, further comprising a braid assembly.

13. The apparatus of claim 1, further comprising a second tendon assembly, the second tendon assembly being operable to drive deflection of a portion of the elongate body away from the central longitudinal axis, the second tendon assembly including:
(i) a second tendon housing extending through the sidewall at a second angular position about the central longitudinal axis, the second tendon housing having a distal end secured to the elongate body, and
(ii) a second tendon slidably disposed in the second tendon housing, the second tendon having a distal portion extending distally from the distal end of the second tendon housing, the distal portion of the second tendon being fixedly secured relative to the elongate body.

14. The apparatus of claim 13, the second angular position being angularly offset from the first angular position by approximately 90 degrees.

15. The apparatus of claim 13, the second angular position being angularly offset from the first angular position by approximately 180 degrees.

16. An apparatus comprising:
(a) an elongate body the elongate body defining a central longitudinal axis, the elongate body including:
(i) a sidewall comprising a flexible material,
(ii) a proximal portion, and
(iii) a distal portion, the distal portion terminating at a distal end;
(b) a tendon assembly extending through the sidewall, the tendon assembly being operable to drive deflection of a portion of the elongate body away from the central longitudinal axis; and
(c) a braid assembly positioned within the sidewall, the braid assembly comprising strands, the tendon assembly being interwoven with the strands such that the tendon assembly is integrated into the braid assembly.

17. The apparatus of claim 16, the tendon assembly including:
(i) a tendon housing extending through the sidewall, the tendon housing having a distal end secured at a longitudinal position along the elongate body, and
(ii) a tendon slidably disposed in the tendon housing, the tendon having a distal portion extending distally from the distal end of the tendon housing, the distal portion of the tendon being fixedly secured relative to the elongate body.

18. A method comprising:
(a) translating a central mandrel relative to a head;
(b) positioning a plurality of outer mandrels about the central mandrel; and
(c) wrapping a plurality of strands about the central mandrel and the outer mandrels to form a braid assembly while the central mandrel translates relative to the head.

19. The method of claim 18, further comprising positioning a liner about the central mandrel, such that the braid assembly is formed about the liner.

20. The method of claim 18, positioning the plurality of outer mandrels about the central lumen including guiding the outer mandrels along the head while the central mandrel translates relative to the head, the plurality of outer mandrels also translating relative to the head while the central mandrel translates relative to the head.
